(19) United States
(12) Patent Application Publication
(10) Pub. No.: US 2023/0161410 A1
(43) Pub. Date: May 25, 2023

(54) Active Device Glove, and Method Enabling Use of Active Technology In Gloves

(71) Applicant: Richard D Ward, Ann Arbor, MI (US)

(72) Inventor: Richard D Ward, Ann Arbor, MI (US)

(21) Appl. No.: 17/756,337

(22) PCT Filed: Sep. 30, 2021
(86) PCT No.: PCT/US2021/071651

§ 371 Date: May 23, 2022

(51) Int. Cl. G06F 3/01; A41D 19/00

(52) U.S. Cl. CPC G06F 3/014; A41D 19/0013; A41D 19/0027; A41D 19/0037

(57) ABSTRACT

The invention moves the wearable device to the area between the thumb and first finger attached or imbedded into the glove to allow immediate access and in just the correct viewing area and angle to allow the user to use without distraction. The options would allow for both a device to be attached like a traditional watch strap or in a specifically designed holder for the specific wearable device. By imbedding the device can allow for contact with the skin therefore enabling sensors to work effectively. This precise location enables gloves for all activities like Biking, Skiing, Workout, CrossFit, Bowling, Golfing, Swimming, Climbing, Baseball, Hockey, Driving, Work Gloves, Etc. to be manufactured with this capability supporting a better way to both use and interact with these wearable devices.

### TECHNICAL FIELD OF THE INVENTION

This invention relates generally to an active technology device enabled glove in the form of a strap, device, or apparatus for attaching an active technology device that is integrated into a glove for the purpose of permitting direct skin contact on the hand for optimized use of said active technology device while needing to wear/use gloves/hand-wear.

The invention relates generally to a sports glove with straps or mounts that secures wearable devices in the optimal position for viewing and interactive usage of a wearable device.

### BACKGROUND

There are gloves designed to permit the visualization of active technology devices and some that include docking connectors that purport to enable use of an active technology device. None allow a user to operate the device or get accurate data from the readings of those devices. The problem is when you typically wear a wearable device like an Apple Watch you fasten it to your wrist. The product Apple Watch is sold under the trademark Apple. Then when doing an activity like skiing you put on a thermal shirt over it, then possibly a fleece, then a Ski Jacket, and finally gloves. When you attempt to access the device, it is either very difficult or not reasonably possible to quickly do without removing clothing. The active glove mounting of the wearable as in the present eliminates this problem.

Attempts to use a strap to attach to the sleeve or arm are not positioning the wearable devices in the best possible location to be truly used. Simply mounting on the back side of a glove also would not be optimal and possibly cause the user to damage the device because of the placement or interference with the given activity.

In light of the foregoing prior art, there is a need for a glove/device that takes into consideration the usage of an active technology device in activities that require the wearing and use of gloves and how potential users interact with their wearable devices to better enables a user see a active technology device and to interact with said active technology device.

### BRIEF SUMMARY OF THE INVENTION

According to a first aspect of the invention there is an Active Device Glove comprising a glove having an active technology device (ATD) mounting and attachment loop comprising a strap having opposing ends attached to said glove by said opposing ends over an area of said glove covering the muscles of thenar eminence in the area between the thumb and index finger of a hand of a wearer enabling a placement of an ATD such that it is maintained in an operationally useful orientation when using said glove.

According to a second aspect of the invention there is an Active Device Glove further comprising an access having an edge through said glove enabling said ATD to be in direct contact with a hand of a wearer wearing said glove.

According to a third aspect of the invention there is an Active Device Glove further comprising an ATD docking connector enabling insertion and removal of said ATD.

According to a fourth aspect of the invention there is an Active Device Glove having an access having and edge therethrough dimensioned to permit direct contact between an ATD and a hand of a wearer wearing said glove located over an area of said glove covering the muscles of thenar eminence in the area between the thumb and index finger of said hand.

According to a first aspect of the invention there is an Active Device Glove comprising a glove, mitten, or sleeve having at least a dorsal hand side, a thumb sleeve, and a forefinger sleeve for the purpose of monitoring a users performance wherein said user has at least a hand having a thumb having at least a first joint and a forefinger comprising an active technology device (ATD) mount having a viewing access for an ATD having a lower left corner and a skin contact access incorporated within said glove positioned on said dorsal hand side between said thumb sleeve and said forefinger sleeve such that said viewing access positions said lower left corner of said ATD in a right angle derived from a line extending from said first joint of said thumb and a line extending from said forefinger, and an option further comprising an ATD docking connector enabling insertion and removal of said ATD.

An advantage of the present invention is that it takes into consideration the usage of an active technology device in activities that require the wearing and use of gloves and how potential users interact with their wearable devices, not to simply see a device but the need to interact with it is as critical an aspect.

The invention will now be described, by way of example only, with reference to the accompanying drawings in which:

### BRIEF DESCRIPTION OF THE DRAWINGS

FIG. 1 is a top view of a glove showing an X over the area of the glove that covers the location of the muscles of thenar eminence in the area between the thumb and index finger of a hand according to the invention;

FIG. 2 is a top view of a glove showing a mounting and attachment loop sewn to glove according to the invention;

FIG. 3 is a top view of a glove showing a mounting and attachment loop sewn to glove holding an active technology device docking connector according to the invention;

FIG. 4 is a top view of a glove showing an active technology device connected to the glove configuration shown in FIG. 3 according to the invention;

FIG. 5 is a top view of a glove showing an access having an edge through said glove (hole through the glove) according to the invention;

FIG. 6 is a top view of a glove showing an access having an edge through said glove (hole through the glove) fitted with an active technology device docking connector according to the invention;

FIG. 7 is a top view of a glove showing use of an active technology device while wearing a bicycling glove according to the invention;

FIG. 8 is a top view of a glove showing use of an active technology device while wearing a bicycling glove according to the invention; and

FIG. 9 is a perspective view from the top of a prototype active technology device docking connector as an Apple Watch holder according to the invention;

FIG. 10 is a perspective view from the bottom of a prototype active technology device docking connector as an Apple Watch holder with an Apple Watch device inserted therein according to the invention.

### DETAILED DESCRIPTION

The detailed embodiments of the present invention are disclosed herein. The disclosed embodiments are merely exemplary of the invention, which may be embodied in various forms. The details disclosed herein are not to be interpreted as limiting, but merely as the basis for the claims and as a basis for teaching one skilled in the art how to make and use the invention.

References in the specification to “one embodiment,” “an embodiment,” “an example embodiment,” etcetera, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures described herein can be spatially arranged in any orientation or manner.

Throughout this specification, the word “comprise”, or variations thereof such as “comprises” or “comprising”, will be understood to imply the inclusion of a stated element, integer or step, or group of elements integers or steps, but not the exclusion of any other element, integer or step, or group of elements, integers or steps.

Throughout this specification, the word “active technology device”, or variations thereof such as “ATD”, will be understood to imply wearable devices and the various smart devices in use by people today with direct human interface technology to include, but not be limited to the Apple Watch, Samsung Galaxy Watch Active, Fossil the Sport Smartwatch, Fitbit devices, as well as other commercially available smart watches and smart devices.

Throughout this specification, the word “docking connector”, or variations thereof, will be understood to imply the inclusion of any active technology device attachment form, template, frame, outline, or sewn-in edge that enables an ATD to be inserted therein and positioned for optimal use orientation.

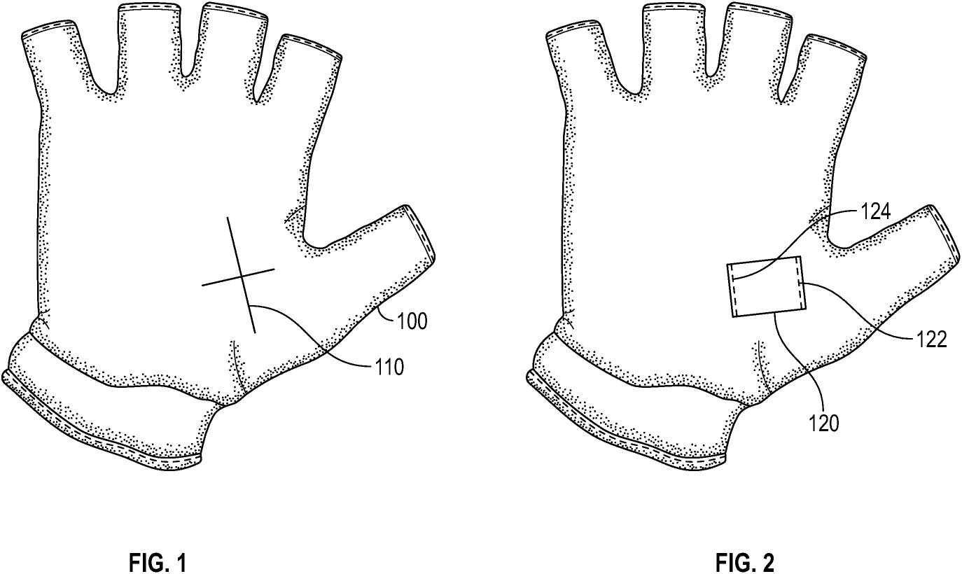

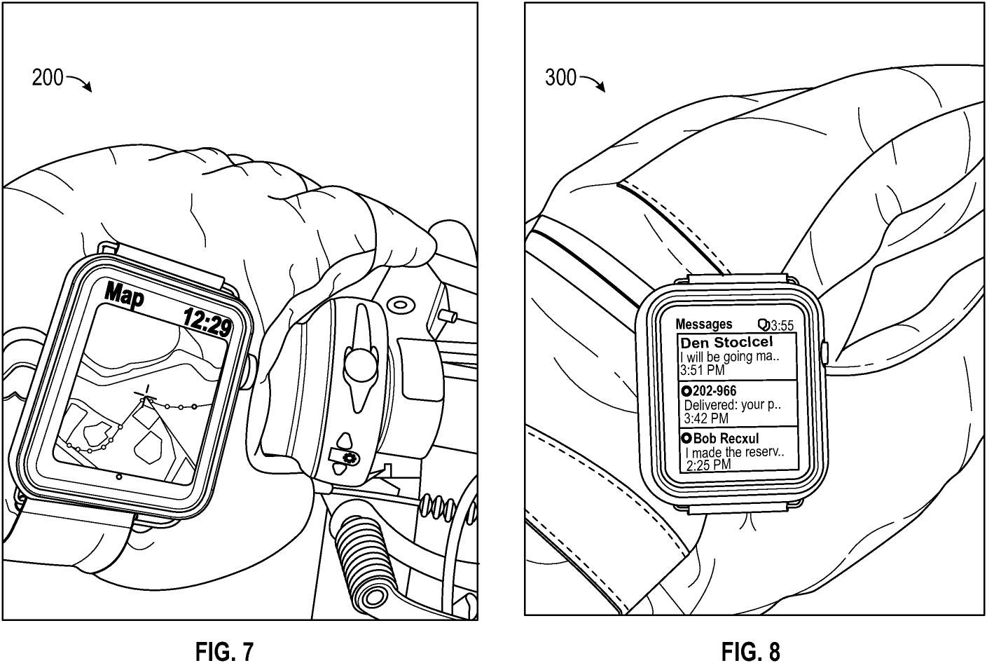

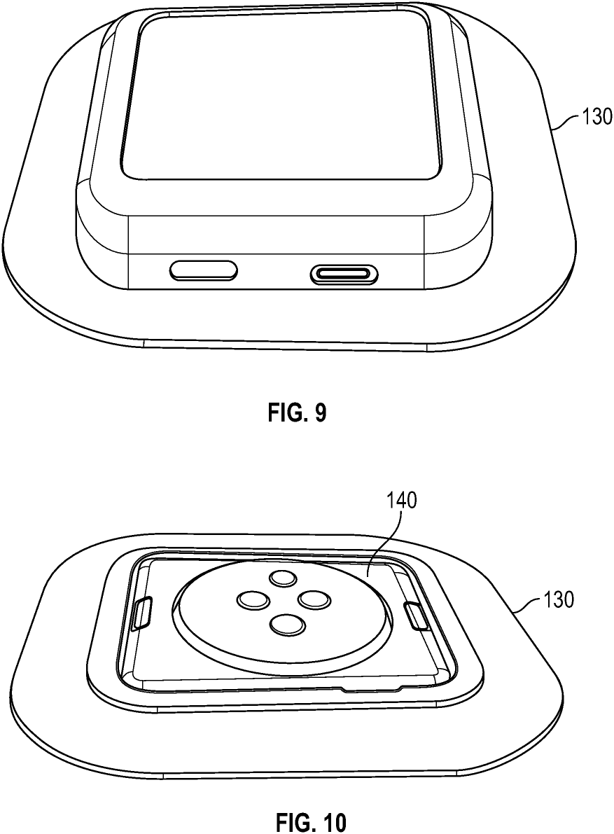

Index of Labelled Features in Figures. Features are listed in numeric order by Figure in numeric order. Referring to the Figures, there is shown in FIGS. 1 through 10 the following features:

Element 100 which is a glove.

Element 110 which is the location of the area of a glove that is over the muscles of thenar eminence of a hand when using said glove.

Element 120 which is a strap or mount that secures wearable devices in the optimal position for viewing and use.

Element 122 and Element 124 are attachments at the ends of the strap 120 to fix said strap 120 to a glove 100.

Element 130 which is an active technology device docking connector.

Element 140 which is an active technology device.

Element 150 which is an access through the glove.

Element 150 which is a mounting or attachment edge for an access through the glove 150.

Element 160 which is an insertable active technology device docking connector.

Element 200 which is a bicycling glove with active technology device attached according to the present invention.

Element 300 which is a skiing glove with active technology device attached according to the present invention.

When you typically wear a wearable device like an Apple Watch you fasten it to your wrist. Then when doing an activity like skiing you put on a thermal shirt over it then possibly a fleece then a Ski Jacket, and gloves. When you attempt to access the device, it is either very difficult or not reasonably possible to quickly do without removing clothing. The active glove mounting of the wearable according to the present invention eliminates this problem.

The first problem was to define the exact placement where a wearable device is most visible, protected from movement and ultimately most accessible. That was outside the clothing and on the glove and aligned on the back of the glove between the first joint of the thumb and index finger. This specific placement will not interfere with the user’s activities and provides the needed accessibility.

The second problem was to design an attachment device that would accommodate a variety of wearables and not require specific gloves for specific devices. A glove with a specific holder is still a desirable outcome however not limited to that requirement. The attachment device as illustrated allows for most devices to be mounted in the specific least active area on the glove and provides acceptable shock absorption from the user’s activities.

Although the strap provides the flexibility of attaching a wide variety of devices it is still preferred to have gloves with a insert that provides both protection and when imbedded into the glove or mitten and will also contact the users skin.

The specific placement of the wearable device is the most critical aspect of the invention. It is also described as area between the thumb and index finger (digitus secundus manus, pointer finger, or forefinger), is called the thenar space. This precise placement provides a secure and unobstructed view of the device and is positioned in a protected area from most active movements. This area is illustrated in FIG. 1 as element 110, the location of the area of a glove that is over the muscles of thenar eminence of a hand when using said glove. Other locations on the outer side of the glove are not ideal and actually interfere with many intended activities.

The present invention is directed to a glove with straps or mounts that secures wearable ATD devices in the optimal position for viewing and use while wearing gloves and actively involved in an activity. The specific location on a glove, mitten or sleeve is critical to the usability. The location is the area between the thumb and forefinger. This is because this is the least active part of the hand and the the least likely to cause an obstruction while in use. The placement of an active technology device is at an angle derived from the first joint of the thumb and forefinger provides the optimum viewing angle maintaining an always in view perspective of the device while performing an activity. This is a key design element. Embedding the device into the glove, mitten or sleeve is the primary solution. This allows the devices sensors to contact the skin and enables the features of active technology device sensors to perform correctly.

By imbedding the device through the glove so contact with the skin is allowed the sensors for pulse, heart rate and oxygen are able to function/operate properly. The present invention is not limited to these sensors however and can accommodate many other options commonly implemented in wearable devices both currently in production or being developed. The Figures show the use of an Apple Watch holder but the present invention is not limited to Apple Devices. Holders (docking connector 130) can be provided for all available wearable devices.

Although it is described as a glove it is not limited to a specific type of glove and or mitten. Full gloves, fingerless gloves, half fingers, sleeves with thumb loops as well as other types of gloves that cover the specific placement area of the back of the hand are covered under this description.

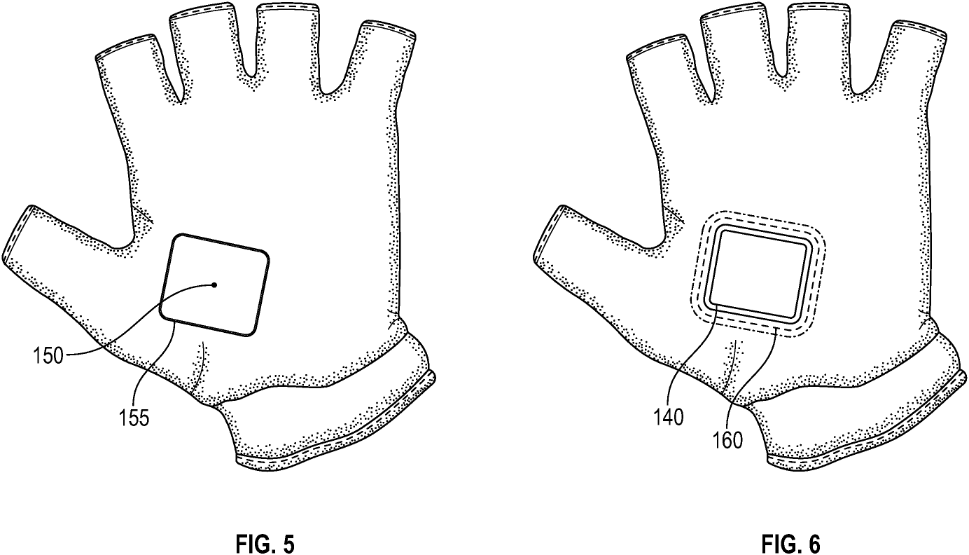

The imbedded wearable holder would be best made of clear TPU but is not limited to this material. Any suitable material commonly used for wearable device protection and or used in the production of athletic equipment or sports gloves/mittens can be used. The holders could be stitched, glued, welded or fused to the underlying material, but is not limited to these methods. Methods commonly used in production of sports equipment or athletic gloves could otherwise be used to attached to the glove/mitten. FIGS. 5 and 6 illustrate the concept of the imbedded device holder (imbedded docking connector 160).

The attachment straps would be made of two components one a flexible webbing material but not limited to this material. Any material approximately 48 mm in width and 40 mm that could be attached to the glove would be possible. This component is attached to the back of the Glove or Mitten in the precise location as previously described. Stitched, glued, welded/fused to the underlying material on each side of the 48 mm with to create a open loop for the second component to be passed through.

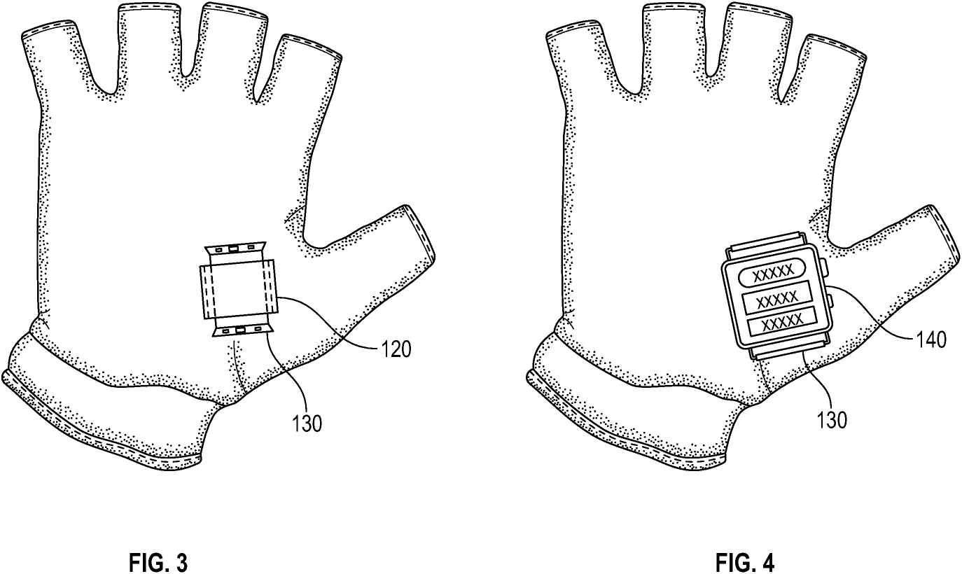

The second component of the strap is made of industrial elastic but not limited to this material, approximately 20 mm wide and 60 mm in length. The second component is sewn at each end to create an open loop that can have an attachment PIN for devices like the Garmin or Apple Watch compatible connector installed. The product Garmin Watch is sold under the trademark Garmin. These connectors are not limited to these but can as well accommodate others that are commonly used in wearable devices. FIG. 3 illustrates the active strap installed with the active loop.

The specific placement of the wearable device is the most critical aspect of the invention. It is also described as area between the thumb and index finger (digitus secundus manus, pointer finger, or forefinger), is called the thenar space. This precise placement provides a secure and unobstructed view of the device and is positioned in a protected area from most active movements.

The imbedded wearable holder would be best made of clear TPU but is not limited to this material. Any suitable material commonly used for wearable device protection and or used in the production of athletic equipment or sports gloves/mittens can be used. The holders could be stitched, glued, welded or fused to the underlying material, but is not limited to these methods.

Methods commonly used in production of sports equipment or athletic gloves could otherwise be used to attached to the glove/mitten.

In a first preferred embodiment, there is a glove having an active technology device (ATD) mounting and attachment loop comprising a strap having opposing ends attached to said glove by said opposing ends over an area of said glove covering the muscles of thenar eminence in the area between the thumb and index finger of a hand of a wearer enabling a placement of an ATD such that it is maintained in an operationally useful orientation when using said glove.

In an alternate embodiment first preferred embodiment, there is a glove further comprising an access having an edge through said glove enabling said ATD to be in direct contact with a hand of a wearer wearing said glove.

In an alternate embodiment first preferred embodiment, there is a glove further comprising an ATD docking connector enabling insertion and removal of said ATD.

In a second preferred embodiment, there is a glove having an access having and edge therethrough dimensioned to permit direct contact between an ATD and a hand of a wearer wearing said glove located over an area of said glove covering the muscles of thenar eminence in the area between the thumb and index finger of said hand.

In an alternate embodiment to the second preferred embodiment, there is a glove further comprising an ATD docking connector enabling insertion and removal of said ATD.

In a preferred embodiment of the invention there is an Active Device Glove comprising a glove, mitten, or sleeve having at least a dorsal hand side, a thumb sleeve, and a forefinger sleeve for the purpose of monitoring a users performance wherein said user has at least a hand having a thumb having at least a first joint and a forefinger comprising an active technology device (ATD) mount having a viewing access for an ATD having a lower left corner and a skin contact access incorporated within said glove positioned on said dorsal hand side between said thumb sleeve and said forefinger sleeve such that said viewing access positions said lower left corner of said ATD in a right angle derived from a line extending from said first joint of said thumb and a line extending from said forefinger, and an option further comprising an ATD docking connector enabling insertion and removal of said ATD.

The invention has been described by way of examples only. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the claims.

Although the invention has been explained in relation to various embodiments, it is to be understood that many other possible modifications and variations can be made without departing from the spirit and scope of the invention.

1-6. (canceled)

7. A glove having at least a dorsal hand side, a thumb sleeve, and a forefinger sleeve for the purpose of monitoring a users performance wherein said user has at least a hand having a thumb having at least a first joint and a forefinger comprising an active technology device (ATD) mount having a viewing access for an ATD having a lower left corner and a skin contact access incorporated within said glove positioned on said dorsal hand side between said thumb sleeve and said forefinger sleeve such that said viewing access positions said lower left corner of said ATD in a right angle derived from a line extending from said first joint of said thumb and a line extending from said forefinger.

8. A glove as in claim 7 further comprising an ATD docking connector enabling insertion and removal of said ATD.

9. A mitten having at least a dorsal hand side, a thumb sleeve, and a forefinger sleeve for the purpose of monitoring a users performance wherein said user has at least a hand having a thumb having at least a first joint and a forefinger comprising an active technology device (ATD) mount having a viewing access for an ATD having a lower left corner and a skin contact access incorporated within said glove positioned on said dorsal hand side between said thumb sleeve and said forefinger sleeve such that said viewing access positions said lower left corner of said ATD in a right angle derived from a line extending from said first joint of said thumb and a line extending from said forefinger.

10. A mitten as in claim 9 further comprising an ATD docking connector enabling insertion and removal of said ATD.

11. A sleeve having at least a dorsal hand side, a thumb sleeve, and a forefinger sleeve for the purpose of monitoring a users performance wherein said user has at least a hand having a thumb having at least a first joint and a forefinger comprising an active technology device (ATD) mount having a viewing access for an ATD having a lower left corner and a skin contact access incorporated within said glove positioned on said dorsal hand side between said thumb sleeve and said forefinger sleeve such that said viewing access positions said lower left corner of said ATD in a right angle derived from a line extending from said first joint of said thumb and a line extending from said forefinger.

12. A sleeve as in claim 11 further comprising an ATD docking connector enabling insertion and removal of said ATD.
